SEQUENCE LISTING (82 sequences)

SEQ ID NO: 1 - DNA, 20 bp, synthetic construct
  source 1..20: mol_type = other DNA
gtatttgtctcccaggtcat

SEQ ID NO: 2 - DNA, 20 bp, synthetic construct
  source 1..20: mol_type = other DNA
acaccacggagcgaagaact

SEQ ID NO: 3 - DNA, 20 bp, synthetic construct
  source 1..20: mol_type = other DNA
tgaccacaccacggagcgaa

SEQ ID NO: 4 - DNA, 20 bp, synthetic construct
  source 1..20: mol_type = other DNA
ccgctttcccttgtactgat

SEQ ID NO: 5 - DNA, 20 bp, synthetic construct
  source 1..20: mol_type = other DNA
ctcagtgactccaaatcccg

SEQ ID NO: 6 - DNA, 20 bp, synthetic construct
  source 1..20: mol_type = other DNA
agtgagtgccaacacagtgc

SEQ ID NO: 7 - DNA, 20 bp, synthetic construct
  source 1..20: mol_type = other DNA
tttcagtgagtgccaacaca

SEQ ID NO: 8 - DNA, 20 bp, synthetic construct
  source 1..20: mol_type = other DNA
aacgagtcaaggtctctgcc

SEQ ID NO: 9 - DNA, 20 bp, synthetic construct
  source 1..20: mol_type = other DNA
gactcgtgatgcaaagctga

SEQ ID NO: 10 - DNA, 20 bp, synthetic construct
  source 1..20: mol_type = other DNA
agactcgtgatgcaaagctg

SEQ ID NO: 11 - DNA, 20 bp, synthetic construct
  source 1..20: mol_type = other DNA
acaagactcgtgatgcaaag

SEQ ID NO: 12 - DNA, 20 bp, synthetic construct
  source 1..20: mol_type = other DNA
ggaatacaagactcgtgatg

SEQ ID NO: 13 - DNA, 16 bp, synthetic construct
  source 1..16: mol_type = other DNA
accacaccacggagcg

SEQ ID NO: 14 - DNA, 16 bp, synthetic construct
  source 1..16: mol_type = other DNA
gctttcccttgtactg

SEQ ID NO: 15 - DNA, 21 bp, synthetic construct
  source 1..21: mol_type = other DNA
aaagttcgtctttcgcttggc

SEQ ID NO: 16 - DNA, 21 bp, synthetic construct
  source 1..21: mol_type = other DNA
tagagcttaagcctgtatgtg

SEQ ID NO: 17 - DNA, 20 bp, synthetic construct
  source 1..20: mol_type = other DNA
aacgtgcagctcaaagttgc

SEQ ID NO: 18 - DNA, 20 bp, synthetic construct
  source 1..20: mol_type = other DNA
gttattttcagtgagtgcca

SEQ ID NO: 19 - DNA, 20 bp, synthetic construct
  source 1..20: mol_type = other DNA
gtctcccaggtcattccaag

SEQ ID NO: 20 - DNA, 20 bp, synthetic construct
  source 1..20: mol_type = other DNA
agccacatagactttggcat

SEQ ID NO: 21 - DNA, 20 bp, synthetic construct
  source 1..20: mol_type = other DNA
cgttcagtgtctgcagcatc

SEQ ID NO: 22 - DNA, 20 bp, synthetic construct
  source 1..20: mol_type = other DNA
tccacttccggctctggtgt

SEQ ID NO: 23 - DNA, 20 bp, synthetic construct
  source 1..20: mol_type = other DNA
aagttgcttgctgagagctc

SEQ ID NO: 24 - DNA, 20 bp, synthetic construct
  source 1..20: mol_type = other DNA
tgtgcaacgtgcagctcaaa

SEQ ID NO: 25 - DNA, 20 bp, synthetic construct
  source 1..20: mol_type = other DNA
acggagcgaagaacttgata

SEQ ID NO: 26 - DNA, 20 bp, synthetic construct
  source 1..20: mol_type = other DNA
ttcaaggcccagagccagct

SEQ ID NO: 27 - DNA, 20 bp, synthetic construct
  source 1..20: mol_type = other DNA
tgttcaaggcccagagccag

SEQ ID NO: 28 - DNA, 20 bp, synthetic construct
  source 1..20: mol_type = other DNA
tagccacgaacctggtttcc

SEQ ID NO: 29 - DNA, 20 bp, synthetic construct
  source 1..20: mol_type = other DNA
ctttcccttgtactgatcca

SEQ ID NO: 30 - DNA, 20 bp, synthetic construct
  source 1..20: mol_type = other DNA
cccgctttcccttgtactga

SEQ ID NO: 31 - DNA, 20 bp, synthetic construct
  source 1..20: mol_type = other DNA
tgactccaaatcccgctttc

SEQ ID NO: 32 - DNA, 20 bp, synthetic construct
  source 1..20: mol_type = other DNA
cagtgagtgccaacacagtg

SEQ ID NO: 33 - DNA, 20 bp, synthetic construct
  source 1..20: mol_type = other DNA
tattttcagtgagtgccaac

SEQ ID NO: 34 - DNA, 20 bp, synthetic construct
  source 1..20: mol_type = other DNA
caagtaggagccagagtctt

SEQ ID NO: 35 - DNA, 20 bp, synthetic construct
  source 1..20: mol_type = other DNA
tcccaagtaggagccagagt

SEQ ID NO: 36 - DNA, 20 bp, synthetic construct
  source 1..20: mol_type = other DNA
agagttcctcccaagtagga

SEQ ID NO: 37 - DNA, 20 bp, synthetic construct
  source 1..20: mol_type = other DNA
tagagagttcctcccaagta

SEQ ID NO: 38 - DNA, 20 bp, synthetic construct
  source 1..20: mol_type = other DNA
actttcttccctcctcggaa

SEQ ID NO: 39 - DNA, 20 bp, synthetic construct
  source 1..20: mol_type = other DNA
tgactttcttccctcctcgg

SEQ ID NO: 40 - DNA, 20 bp, synthetic construct
  source 1..20: mol_type = other DNA
tctctgcctccactgtgctc

SEQ ID NO: 41 - DNA, 20 bp, synthetic construct
  source 1..20: mol_type = other DNA
aggtctctgcctccactgtg

SEQ ID NO: 42 - DNA, 20 bp, synthetic construct
  source 1..20: mol_type = other DNA
cgagtcaaggtctctgcctc

SEQ ID NO: 43 - DNA, 20 bp, synthetic construct
  source 1..20: mol_type = other DNA
ccacatagactttggcatct

SEQ ID NO: 44 - DNA, 20 bp, synthetic construct
  source 1..20: mol_type = other DNA
ccacggagcgaagaacttga

SEQ ID NO: 45 - DNA, 20 bp, synthetic construct
  source 1..20: mol_type = other DNA
atcccgctttcccttgtact

SEQ ID NO: 46 - DNA, 20 bp, synthetic construct
  source 1..20: mol_type = other DNA
tcccaggtcattccaagtcg

SEQ ID NO: 47 - DNA, 20 bp, synthetic construct
  source 1..20: mol_type = other DNA
caaatcccgctttcccttgt

SEQ ID NO: 48 - DNA, 20 bp, synthetic construct
  source 1..20: mol_type = other DNA
tccacgtactccctcagtga

SEQ ID NO: 49 - DNA, 20 bp, synthetic construct
  source 1..20: mol_type = other DNA
agtctctgtgcgctgcagct

SEQ ID NO: 50 - DNA, 20 bp, synthetic construct
  source 1..20: mol_type = other DNA
cagtctacttcggcgatctt

SEQ ID NO: 51 - DNA, 20 bp, synthetic construct
  source 1..20: mol_type = other DNA
aagcggtgtaacgagtcaag

SEQ ID NO: 52 - DNA, 20 bp, synthetic construct
  source 1..20: mol_type = other DNA
gtgaaccatacgtgattaat

SEQ ID NO: 53 - DNA, 20 bp, synthetic construct
  source 1..20: mol_type = other DNA
ggaacgtcaatactgccaca

SEQ ID NO: 54 - DNA, 20 bp, synthetic construct
  source 1..20: mol_type = other DNA
gcttcgtgttaacatgagga

SEQ ID NO: 55 - DNA, 20 bp, synthetic construct
  source 1..20: mol_type = other DNA
gaactctagttagggccctt

SEQ ID NO: 56 - DNA, 20 bp, synthetic construct
  source 1..20: mol_type = other DNA
tccagaactcgtgggcaggt

SEQ ID NO: 57 - DNA, 20 bp, synthetic construct
  source 1..20: mol_type = other DNA
aggatcccctgaacagtaac

SEQ ID NO: 58 - DNA, 20 bp, synthetic construct
  source 1..20: mol_type = other DNA
ggctctggcttcgtgttaac

SEQ ID NO: 59 - DNA, 20 bp, synthetic construct
  source 1..20: mol_type = other DNA
agaactctagttagggccct

SEQ ID NO: 60 - DNA, 20 bp, synthetic construct
  source 1..20: mol_type = other DNA
tgggccaagtatccacatcc

SEQ ID NO: 61 - DNA, 20 bp, synthetic construct
  source 1..20: mol_type = other DNA
gccacctttccagaactcgt

SEQ ID NO: 62 - DNA, 20 bp, synthetic construct
  source 1..20: mol_type = other DNA
actgagaatgagcctcgtgg

SEQ ID NO: 63 - DNA, 20 bp, synthetic construct
  source 1..20: mol_type = other DNA
cctgggaaagcctgtctggt

SEQ ID NO: 64 - DNA, 20 bp, synthetic construct
  source 1..20: mol_type = other DNA
gcctccctggatacccaggc

SEQ ID NO: 65 - DNA, 20 bp, synthetic construct
  source 1..20: mol_type = other DNA
gacatctgtctttggtcttg

SEQ ID NO: 66 - DNA, 20 bp, synthetic construct
  source 1..20: mol_type = other DNA
ggacagcatgagttagagga

SEQ ID NO: 67 - DNA, 20 bp, synthetic construct
  source 1..20: mol_type = other DNA
cctaccaccagttactttgg

SEQ ID NO: 68 - DNA, 20 bp, synthetic construct
  source 1..20: mol_type = other DNA
cttgacagttgtgcttctct

SEQ ID NO: 69 - DNA, 20 bp, synthetic construct
  source 1..20: mol_type = other DNA
aagttgtgtgtacaagactc

SEQ ID NO: 70 - DNA, 20 bp, synthetic construct
  source 1..20: mol_type = other DNA
  misc_feature 1..5: note = 2'-MOE modified sugar
  misc_feature 16..20: note = 2'-MOE modified sugar
gtatttgtctcccaggtcat

SEQ ID NO: 71 - DNA, 20 bp, synthetic construct
  source 1..20: mol_type = other DNA
  misc_feature 1..5: note = 2'-MOE modified sugar
  misc_feature 16..20: note = 2'-MOE modified sugar
acaccacggagcgaagaact

SEQ ID NO: 72 - DNA, 20 bp, synthetic construct
  source 1..20: mol_type = other DNA
  misc_feature 1..5: note = 2'-MOE modified sugar
  misc_feature 16..20: note = 2'-MOE modified sugar
tgaccacaccacggagcgaa

SEQ ID NO: 73 - DNA, 20 bp, synthetic construct
  source 1..20: mol_type = other DNA
  misc_feature 1..5: note = 2'-MOE modified sugar
  misc_feature 16..20: note = 2'-MOE modified sugar
ccgctttcccttgtactgat

SEQ ID NO: 74 - DNA, 20 bp, synthetic construct
  source 1..20: mol_type = other DNA
  misc_feature 1..5: note = 2'-MOE modified sugar
  misc_feature 16..20: note = 2'-MOE modified sugar
ctcagtgactccaaatcccg

SEQ ID NO: 75 - DNA, 20 bp, synthetic construct
  source 1..20: mol_type = other DNA
  misc_feature 1..5: note = 2'-MOE modified sugar
  misc_feature 16..20: note = 2'-MOE modified sugar
agtgagtgccaacacagtgc

SEQ ID NO: 76 - DNA, 20 bp, synthetic construct
  source 1..20: mol_type = other DNA
  misc_feature 1..5: note = 2'-MOE modified sugar
  misc_feature 16..20: note = 2'-MOE modified sugar
tttcagtgagtgccaacaca

SEQ ID NO: 77 - DNA, 20 bp, synthetic construct
  source 1..20: mol_type = other DNA
  misc_feature 1..6: note = 2'-MOE modified sugar
  misc_feature 16..20: note = 2'-MOE modified sugar
aacgagtcaaggtctctgcc

SEQ ID NO: 78 - DNA, 20 bp, synthetic construct
  source 1..20: mol_type = other DNA
  misc_feature 1..5: note = 2'-MOE modified sugar
  misc_feature 16..20: note = 2'-MOE modified sugar
gactcgtgatgcaaagctga

SEQ ID NO: 79 - DNA, 20 bp, synthetic construct
  source 1..20: mol_type = other DNA
  misc_feature 1..5: note = 2'-MOE modified sugar
  misc_feature 16..20: note = 2'-MOE modified sugar
agactcgtgatgcaaagctg

SEQ ID NO: 80 - DNA, 20 bp, synthetic construct
  source 1..20: mol_type = other DNA
  misc_feature 1..5: note = 2'-MOE modified sugar
  misc_feature 16..20: note = 2'-MOE modified sugar
acaagactcgtgatgcaaag

SEQ ID NO: 81 - DNA, 20 bp, synthetic construct
  source 1..20: mol_type = other DNA
  misc_feature 1..5: note = 2'-MOE modified sugar
  misc_feature 16..20: note = 2'-MOE modified sugar
ggaatacaagactcgtgatg

SEQ ID NO: 82 - DNA, 20 bp, synthetic construct
  source 1..20: mol_type = other DNA
  misc_feature 1..5: note = 2'-MOE modified sugar
  misc_feature 16..20: note = 2'-MOE modified sugar
gtctcacgctactgttcttc

(19) United States
(12) Patent Application Publication
(10) Pub. No.: US 2025/0066793 A1
(43) Pub. Date: Feb. 27, 2025

(54) ANTI-SENSE OLIGONUCLEOTIDES AND USES THEREOF

(71) Applicant: Development Center for Biotechnology, New Taipei City (TW)

(72) Inventors: Ying-Shuan LAILEE, New Taipei City (TW); Chia-Wei LIU, New Taipei City (TW); Chi-Tang WANG, New Taipei City (TW); Pei-Yi TSAI, New Taipei City (TW); Chung-Hsiun WU, New Taipei City (TW); King LAM, New Taipei City (TW); Wei-Ting SUN, New Taipei City (TW); Kai-Chien YANG, New Taipei City (TW); Hung-Jyun HUANG, New Taipei City (TW)

(21) Appl. No.: 18/724,758

(22) PCT Filed: Dec. 28, 2022
(86) PCT No.: PCT/US2022/082445

§ 371 Date: Jun. 27, 2024

Related U.S. Application Data
(60) Provisional application No. 63/294,835, filed on Dec. 29, 2021.

(51) Int. Cl. C12N 15/113; A61P 11/00

(52) U.S. Cl. CPC C12N 15/1137; A61P 11/00; C12Y 503/04001; C12N 2310/11; C12N 2310/321; C12N 2310/322; C12N 2310/3231

(57) ABSTRACT

Disclosed herein are novel single-stranded anti-sense oligonucleotides (ASOs) capable of reducing the transcription of thioredoxin domain containing protein 5 (TXNDC5) mRNA. Also disclosed is use of the single-stranded ASOs as disclosed herein for manufacturing medicaments suitable for treating a disease associated with upregulation of TXNDC5. Accordingly, a pharmaceutical composition comprising the disclosed ASO molecules is provided; as well as a method of treating a subject suffering from TXNDC5-mediated disease via administering to the subject the disclosed single-stranded ASO molecules.

### CROSS-REFERENCE TO RELATED APPLICATIONS

This application claims priority and the benefit of U.S. Provisional Patent Application No. 63/294,835, filed Dec. 29, 2021, the entirety of which is incorporated herein by reference.

### BACKGROUND OF THE INVENTION

### 1. Field of the Invention

The present disclosure relates to treatments of disease. More particularly, the disclosure relates to treating diseases by use of single-stranded anti-sense oligonucleotides (ASO) that inhibit the expression of thioredoxin domain containing protein 5 (TXNDC5) mRNA.

### 2. Description of Related Art

The thioredoxin domain containing protein 5 (TXNDC5) is a protein-disulfide isomerase that catalyzes its thioredoxin activity and enable it to act as a chaperon in the endoplasmic reticulum. Upregulated expression of TXNDC5 have been observed to be associated with various types of diseases, including cancers, diabetes, arthritis, neurodegenerative disease, organ fibrosis related disease (e.g., pulmonary fibrosis, kidney fibrosis, liver fibrosis, and myocardial fibrosis), and vitiligo.

With the emerging use of post-transcriptional gene silencing technology, in particular, antisense oligonucleotide (i.e., ASO), as a tool to knock out expression of specific genes in a variety of organisms, it is now possible to map protein interactions in cell signaling pathway by systematically silencing functional genes, and thereby providing a new way of developing therapeutics for countless diseases. After extensive researches and experiments, inventors of the present study have identified short oligonucleotide molecules capable of targeting pre-mRNA or mRNA of TXNDC5, and reducing the TXNDC5 RNA level, which in turn lowering the TXNDC5 protein level. Antisense molecules can act on a target sequence through various mechanisms of action: degradation of mRNA through RNaseH, steric hindrance of ribosomal subunit binding, altering maturation of mRNA, 5′-cap formation inhibition, arrest of translation. Thus, these identified short nucleic acid molecules are useful for the development of a medicament for treating diseases associated with overexpression of TXNDC5, thereby alleviating or minimizing symptoms associated therewith in subjects in need of such treatment.

### SUMMARY

The present disclosure is directed to single-stranded nucleic acids for treatment of, or prophylaxis against, diseases, particularly, diseases that are associated with upregulation of TXNDC5.

Accordingly, one aspect of the present invention is directed to an isolated single-stranded anti-sense oligonucleotide (ASO) that reduces the TXNDC5 mRNA expression, wherein said ASO molecule is about 16 to 21 nucleotides in length, and comprises a deoxyribonucleotide sequence having at least 80% sequence identity to any of SEQ ID NOs: 1, 2, 3, 4, 5, 6, 7, 8, 9, 10, 11, 12, 13 or 14.

According to preferred embodiments of the present disclosure, said ASO molecule comprises a deoxyribonucleotide sequence having at least 90% sequence identity to any of SEQ ID NOs: 1, 2, 3, 4, 5, 6, 7, 8, 9, 10, 11, 12, 13 or 14.

According to embodiments of the present disclosure, said ASO molecule comprises at least one locked nucleic acid (LNA) molecule, 2′-sugar modification, modified internucleotide linkage or a combination thereof. In some embodiments of the present disclosure, the ASO molecule comprises 6 LNA molecules. In other embodiments of the present disclosure, the ASO molecule comprises 10 2′-sugar modifications.

In another aspect, the present disclosure is directed to a method of treating a subject suffering from a disease that is mediated through upregulation of TXNDC5. The method comprises the step of administering to the subject a therapeutically effective amount of the ASO molecule of this invention to suppress the transcription of TXNDC5 gene.

According to embodiments of the present disclosure, the ASO molecule of this invention is a single-stranded oligonucleotide that reduces TXNDC5 mRNA expression. Said ASO molecule is about 16 to 21 nucleotides in length, and has a deoxyribonucleotide sequence having at least 80% sequence identity to any of SEQ ID NOs: 1, 2, 3, 4, 5, 6, 7, 8, 9, 10, 11, 12, 13 or 14.

According to embodiments of the present disclosure, the ASO molecule comprises at least one LNA molecule, 2′-sugar modification, modified internucleotide linkage or a combination thereof. In some embodiments, the ASO molecule comprises 6 LNA molecules. In other embodiments, the ASO molecule comprises 10 2′-sugar modifications.

According to embodiments of the present disclosure, the disease associated with overexpression of TXNDC5 is selected from the group consisting of aging, arthritis (e.g., rheumatoid arthritis), cancer, diabetes (e.g., Type II diabetes), neurodegenerative disease, fibrosis, vitiligo, and virus infection. In one preferred example, the subject is suffering from organ fibrosis such as pulmonary fibrosis, kidney fibrosis, liver fibrosis, or myocardial fibrosis.

In another aspect, the present disclosure is directed to a pharmaceutical composition for treating, preventing, or ameliorating a disease associated with overexpression of TXNDC5. The pharmaceutical composition comprises the ASO molecule of this invention; and a pharmaceutically acceptable carrier.

The details of one or more embodiments of the invention are set forth in the accompanying description below. Other features and advantages of the invention will be apparent from the detail descriptions, and from claims.

### BRIEF DESCRIPTION OF THE DRAWINGS

These and other features, aspects and advantages of the present invention will become better understood with reference to the following description, appended claims, and the accompanying drawings, where:

FIGS. 1A to 1I illustrate the effect of the present ASO-MOEs or ASO-LNAs on expression patterns of TXNDC5 and fibrosis related proteins with or without TGF-β induction via western blot analysis in accordance with some embodiments of this invention, the present ASOs are respectively (A) DCB11111128235, (B) DCB11111128255, (C) DCB1111128252, (D) DCB1111128266, (E) DCB1111128265, (F) DCB1111128238, (G) DCB1111128279, (H) DCB1111128280, and (I) DCB1111128281;

FIGS. 2A to 2C illustrate the effect of the present ASO-MOEs on lung function of mice with pulmonary fibrosis induced by Bleomycin (BLM) in accordance with one embodiment of this invention, in which (A) is Compliance factor, (B) is Resistance factor, and (C) is Elastance factor of the lung function;

FIG. 3 illustrates the Pressure-Volume curves in Bleomycin-induced fibrotic mice in accordance with one embodiment of this invention; and

FIG. 4 illustrates the effect of the present ASO-MOEs on reducing fibrotic area in BLM-induced fibrotic mice in accordance with one embodiment of this invention.

### DESCRIPTION

The detailed description provided below in connection with the appended drawings is intended as a description of the present examples and is not intended to represent the only forms in which the present examples may be constructed or utilized. The description sets forth the functions of the invention and the sequence of steps for constructing and operating the examples. However, the same or equivalent functions and sequences may be accomplished by different examples.

### 1. Definitions

For convenience, certain terms employed in the context of the present disclosure are collected here. Unless defined otherwise, all technical and scientific terms used herein have the same meaning as commonly understood by one of the ordinary skilled persons in the art to which this invention belongs.

The term “nucleic acid” is defined as a molecule formed by covalent linkage of two or more nucleotides, which encompass DNA, RNA, and variants or analogues of such DNA or RNA. The terms “nucleic acid” and “polynucleotide” are used interchangeable herein. The term “oligonucleotide” is defined as a nucleic acid that consists of less than 25 nucleotides, such as 20 nucleotides.

Antisense oligonucleotides (ASO) as used herein refers to single stranded DNA or RNA that are complementary to a pre-mRNA or mRNA sequence, and can reduce the RNA level thereby reducing the protein level. According to preferred embodiments of the present disclosure, ASOs complementary to nucleobases at positions 337 to 356, 670 to 689, 675 to 694, 862 to 881, 879 to 898, 1003 to 1022, 1007 to 1026, 1278 to 1297, 2864 to 2883, 2865 to 2884, 2868 to 2887 and 2873 to 2892 of TXNDC5 mRNA (NCBI reference sequence: NM_030810.4) are produced and result in down-regulation of TXNDC5 mRNA.

As used herein, a “sequence” of a nucleic acid refers to the ordering of nucleotides which make up a nucleic acid. Throughout this application, nucleic acids are designated as having a 5′ end and a 3′ end. Unless specified otherwise, the left-hand end of a single-stranded nucleic acid is the 5′ end; and the right-hand end of single-stranded nucleic acid is the 3′ end.

The term “lock nucleic acid (LNA)” as used herein refers to a nucleic acid in which some nucleotides of the nucleic acid are lock nucleic acid monomers (i.e., bicyclic nucleotide or its analogues). The ribose moiety of an LNA nucleotide is modified with an extra bridge connecting the 2′-oxygen and 4′-carbon. The bridge “locks” the ribose in the 3′-endo (North) conformation, which is often found in the A-form duplexes. These LNA monomers are described inter alia in WO 2001/25248, WO 2003/006475 and WO 2003/095467; disclosures of the respective recited publications are incorporated herein by reference.

The term “complementary” refers to polynucleotides (i.e., a sequence of nucleotides) related by the base-pairing rule. For example, the sequence “A-G-T,” is complementary to the sequence of “T-C-A.” Polynucleotides are described as “complementary” to one another when hybridization occurs in an antiparallel configuration between two single-stranded polynucleotides.

“Percentage (%) sequence identity” with respect to any nucleotide sequence identified herein is defined as the percentage of nucleotide residues in a candidate sequence that are identical with the nucleotide residues in the specific nucleotide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two nucleotide sequences was carried out by computer program Blastn (nucleotide-nucleotide BLAST) provided online by Nation Center for Biotechnology Information (NCBI). The percentage sequence identity of a given nucleotide sequence A to a given nucleotide sequence B (which can alternatively be phrased as a given nucleotide sequence A that has a certain % nucleotide sequence identity to a given nucleotide sequence B) is calculated by the formula as follows:

$$\frac{X}{Y} \times 100 \%$$

where X is the number of nucleotide residues scored as identical matches by the sequence alignment program BLAST in that program's alignment of A and B, and where Y is the total number of nucleotide residues in A or B, whichever is shorter.

As used herein, the terms “treat” or “treating” or “treatment” refer to preventative (e.g., prophylactic), curative or palliative treatment. The term “treating” as used herein refers to application or administration of the ASO of the present disclosure to a subject, who has a medical condition, a symptom of the condition, a disease or disorder secondary to the condition, or a predisposition toward the condition, with the purpose to partially or completely alleviate, ameliorate, relieve, delay onset of, inhibit progression of, reduce severity of, and/or reduce incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable.

The term “effective amount” as used herein refers to the quantity of a component which is sufficient to yield a desired response. The term “therapeutically effective amount” as used herein refers to the amount of therapeutically agent (e.g., the present ASO) to result in a desired “effective treatment” as defined hereinabove. The specific therapeutically effective amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed. A therapeutically effective amount is also one in which any toxic or detrimental effects of the compound or composition are outweighed by the therapeutically beneficial effects.

The term “subject” or “patient” as used herein refers to a human or a non-human animal with a dysregulated expression of TXNDC5, particularly, upregulation of TXNDC5 as compared to that of a healthy subject, and subject to methods of the present invention. The term “subject” or “patient” intended to refer to both the male and female gender unless one gender is specifically indicated. Examples of a non-human animal include all vertebrates, e.g., mammals, such as primates, dogs, rodents (e.g., mouse or rat), cats, sheep, horses or pigs; and non-mammals, such as birds, amphibians, and etc.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

### 2. The Present Anti-Sense Oligonucleotides (ASOs)

The present disclosure is directed to a novel solution for treating disease associated with upregulated TXNDC5 by use of an anti-sense oligonucleotide (ASO) molecule. Accordingly, in its broadest aspect, the present invention relates to a single-stranded deoxyribonucleic acid complementary to at least a portion of a target gene such as TXNDC5 mRNA, and when transfected into host cells, said single-stranded deoxyribonucleic acid is capable of suppressing the expression of the target gene mRNA.

The single-stranded deoxyribonucleic acid or the ASO molecule of this invention is about 16 to 21 nucleotides in length; and comprises a deoxyribonucleotide sequence having at least 80% sequence identity to any of SEQ ID NOs: 1, 2, 3, 4, 5, 6, 7, 8, 9, 10, 11, 12, 13 or 14. According to preferred embodiments of the present disclosure, said ASO molecule comprises a deoxyribonucleotide sequence having at least 90% sequence identity to any of SEQ ID NOs: 1, 2, 3, 4, 5, 6, 7, 8, 9, 10, 11, 12, 13 or 14. In certain preferred embodiments, said ASO molecule comprises a deoxyribonucleotide sequence 100% identical to SEQ ID No: 1, 2, 3, 4, 5, 6, 7, 8, 9, 10, 11, 12, 13 or 14. The ASO of the present disclosure may be 16 to 21 nucleotides in length, such as 16, 17, 18, 19, 20, or 21 nucleotides in length. In some preferred examples, the ASO of the present disclosure has 20 nucleotides in length. In other preferred examples, the ASO of the present disclosure has 16 nucleotides in length.

### 3. Modified ASO

Alternatively, or in addition, the present ASO may contain modifications at its backbone nucleobases (e.g., substitutions) or at its sugar rings, such as by introducing substitutions or changes to internucleotide linkages, sugar moieties, or nucleobases. In some embodiments, the nucleic acid of the present ASO is composed of DNA molecules in combination with one or more LNA molecules. In othe rembodiments, the nucleic acid of the present ASO is composed of DNA molecules in combination with one or more 2′-sugar modifications (e.g., 2′-O-methoxyethyl substitution). Modified ASOs are often preferred over native forms because of desirable properties such as, for example, enhanced cellular uptake, enhanced affinity for nucleic acid target, increased stability in the presence of nucleases, or increased inhibitory activity.

### (i) Modified Internucleotide Linkage

The naturally occurring internucleotide linkage of RNA and DNA is a 3′ to 5′ phosphodiester linkage. Oligonucleotides having modified internucleotide linkages include internucleotide linkages that retain a phosphorus atom as well as internucleotide linkages that do not have a phosphorus atom. Representative phosphorus containing internucleotide linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, the present ASO comprises one or more modified internucleotide linkages, such as one or more phosphorothioate linkages.

### (ii) Modified Sugar Moiety

The present ASO may optionally contain one or more nucleosides, in which the sugar group has been modified. In certain embodiments, the present ASO comprises a chemically modified ribofuranose ring moiety. Examples of the chemically modified ribofuranose rings include without limitation, addition of substitutent groups (e.g., 5′- or 2′-substituent groups), bridging of non-geminal ring atoms to form lock nucleic acids (LNAs), replacement of the ribosyl ring oxygen atom with S, N(R), or C(Rₐ)(R_{b})₂, in which R, Rₐ, and R_{b} are independently C₁₋₁₂ alkyl, a protecting group, or a combination thereof.

Examples of chemically modified sugars include 2′-F-5′-methyl substituted nucleoside or replacement of the ribosyl ring oxygen atom with S with further substitution at the 2′-position.

According to alernative examples, the chemically modified sugars include 5′-substitution of a LNA molecule. Examples of LNAs include without limitation nucleosides comprising a bridge between the 4′ and the 2′ ribosyl ring atoms. In certain embodiments, the present ASO herein include one or more LNA nucleosides wherein the bridge comprises one of the formulas: 4′-(CH₂)—O-2′ (LNA); 4′-(CH₂)—S-2; 4′-(CH₂)—O-2′ (LNA); 4′-(CH₂)₂—O-2′ (ENA); 4′-C(CH₃) 2-O-2′; 4′-CH(CH₃)—O-2′ and 4′-CH(CH₂OCH₃)—O-2′; 4′-CH₂—N(OCH₃)-2′; 4′-CH₂—O—N(CH₃)-2′; 4′—CH₂—NR—O-2′; 4′-CH₂—C(CH₃)-2′ and 4′-CH₂—C(═CH₂)-2′, wherein R is independently, H, C₁₋₁₂ alkyl, or a protecting group. Each of the foregoing LNAs include various stereochemical sugar configurations including for example α-L-ribofuranose and β-D-ribofuranose.

In certain embodiments, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the following formula:

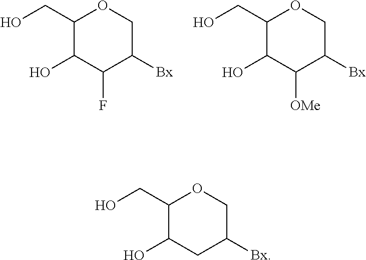

Many other bicyclo and tricyclo sugar surrogate ring systems are also known in the art that can be used to modify nucleosides for incorporation into the present ASO.

Accordingly, the present ASO may be composed of entirely DNA molecules or it may be composed of DNA molecules in combination with at least one modified nucleotide. In some embodiments, the present ASO is composed of DNA molecules in combination of LNA molecule such as 2′-O—, 4′-C methylene bicyclonucleoside monomer. One advantage of having LNA monomer(s) in a nucleic acid is that the stability of nucleic acid is improved; accordingly, the ASO of this invention may include the incorporation of LNA monomers into a standard DNA oligonucleotide to increase the stability of the resulting molecule, such as increasing the resistance of ASO toward protease (endonucleases and exonucleases) and thereby increasing its circulating half-life in a biological sample. In general, the single-stranded ASO of this invention may contain at least about 5%, 10%, 15%, 20%, 25% or 30% LNA monomers, based on total number of nucleotides in the strand. In certain embodiments, the ASO of this invention will contain at least about 25%, 30%, 40%, 50% or 60% LNA monomers, based on total number of nucleotides in the strand; preferably about 40% LNA monomers; and more preferably about 60% LNA monomers. In one embodiment of this invention, the ASO of this invention is composed of entirely DNA molecules, in which one strand comprises a nucleotide sequence having at least 90% sequence identity, such as 90%, 91%, 92%, 93%, 94%, 95%, 96%, 97%, 98%, 99% or 100%, to any of SEQ ID NOs: 1, 2, 3, 4, 5, 6, 7, 8, 9, 10, 11, 12, 13 or 14. In another embodiment of this invention, the ASO of this invention is composed of DNA molecules in combination with LNA molecules, in which the single-stranded ASO is composed of DNA molecules in combination with at least about 30% LNA molecules. In one embodiment of this invention, the single-stranded ASO comprises at least one LNA monomer. In another embodiment, the single-stranded ASO comprises six LNA monomers.

Alternatively, the single-stranded ASO of the invention may be composed of DNA molecules in combination with at least one 2′-sugar modification, such as 2′-O-methoxyethyl (2′-O-MOE) modified sugar. In general, the individual strand of the single-stranded ASO of this invention may contain at least about 5%, 10%, 15%, 20%, 25% or 30% 2′-sugar modification, based on total number of nucleotides in the strand. In certain embodiments, the single-stranded ASO of this invention will contain at least about 25%, 30%, 40%, 50% or 60% 2′-sugar modification, based on total number of nucleotides in the strand; preferably about 40% 2′-sugar modification; and more preferably about 50% 2′-sugar modification. In one embodiment of this invention, the single-stranded ASO of this invention is composed of entirely DNA molecules and comprises a nucleotide sequence having at least 90% sequence identity, such as 90%, 91%, 92%, 93%, 94%, 95%, 96%, 97%, 98%, 99% or 100%, to any of SEQ ID NOs: 1, 2, 3, 4, 5, 6, 7, 8, 9, 10, 11, 12, 13 or 14. In another embodiment of this invention, the single-stranded ASO of this invention is composed of DNA molecules in combination with one or more 2′-sugar modifications, in which the strand of deoxyribonucleic acid is composed of DNA molecules in combination with at least 20% 2′-sugar modification. In one embodiment of this invention, the single-stranded ASO comprises only one 2′-O-MOE modified sugar. In another embodiment, the single-stranded ASO comprises ten 2′-O-MOE modified sugars.

(iii) Modified Nucleobases

Chemically modified nucleosides may also be included in the present ASO to increase its binding affinity to the target nucleic acid. Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense compounds. Modified nucleobases include synthetic and natural nucleobases such as, 5-methylcytosine (5-me-C). Certain nucleobase substitutions, including 5-methylcytosine substitutions, are particularly useful for increasing the binding affinity of an antisense oligomer to a target nucleic acid.

Accordingly, the present single-stranded ASO may be composed of DNA molecules in combination with at least one modified nucleobase, such as 5-methylcytosine, 5-hydroxymethylcytosine; xanthine; hypoxanthine; 2-aminoadenine; 6-methyl and other alkyl derivatives of adenine and guanine; 2-propyl and other alkyl derivatives of adenine and guanine; 2-thiouracil; 2-thiothymine; 2-thiocytosine; 5-halouracil; 5-halocytosine; 5-propynyl uracil and cytosine and other alkynyl derivatives of pyrimidine bases; 6-azo uracil, cytosine and thymine; 5-uracil; 4-thiouracil; 8-halo, 8-amino, 8-thiol, 8-thioalkyl, 8-hydroxyl and other 8-substituted adenines and guanines; 5-halo particularly 5-bromo, 5-trifluoromethyl and other 5-substituted uracils and cytosines; 7-methylguanine; 7-methyladenine; 2-F-adenine; 2-amino-adenine; 8-azaguanine; 8-azaadenine; 7-deazaguanine; 7-deazaadenine; 3-deazaguanine; and 3-deazaadenine. In general, the single-stranded ASO of this invention may contain at least about 5%, 10%, 15%, 20%, 25% or 30% modified nucleobase, based on total number of nucleotides in the strand. In certain embodiments, the single-stranded ASO of this invention contains at least about 25%, 30%, 40%, 50% or 60% modified nucleobases, based on total number of nucleotides in the strand; preferably about 40% modified nucleobases; and more preferably about 50% modified nucleobases. In one embodiment of this invention, the single-stranded ASO of this invention is composed of entirely DNA molecules and comprises a nucleotide sequence having at least 90% sequence identity, such as 90%, 91%, 92%, 93%, 94%, 95%, 96%, 97%, 98%, 99% or 100%, to any of SEQ ID NOs: 1, 2, 3, 4, 5, 6, 7, 8, 9, 10, 11, 12, 13 or 14. In another embodiment of this invention, the single-stranded ASO of this invention is composed of DNA molecules in combination with 5-methylcytosine (e.g., at least 20% 5-methylcytosine). In some embodiments of this invention, the single-stranded ASO comprises only one 5-methylcytosine. In other embodiments, the single-stranded ASO comprises ten 5-methylcytosines.

### 4. Methods of Producing the Present ASOs

Currently, there are various ways for generating ASO with or without chemical modification described above for gene silencing studies, including chemical synthesis, or digestion of long dsDNA by a DNase III family enzyme. These methods involve in vitro preparation of nucleic acids that are then introduced directly into cells by lipofection, electroporation or other technique.

The ASO molecules of this invention were obtained by in vitro preparation and/or chemical synthesis using protocols known in the art. For example, the single-stranded nucleic acid of this invention may be produced using the polymerization techniques of nucleic acid chemistry, which is well known to a person of ordinary skill in the art of organic chemistry. In general, standard oligomerization cycles of the phosphoramidite approach may be used, but other chemistries, such as H-phosphonate chemistry or phosphotriester chemistry may also be used. The present ASO molecules may be delivered to a subject directly, or via a delivery vehicle such as liposomes. In addition, the present ASO may be formulated with suitable carriers, and/or diluents to give pharmaceutically acceptable formulations. Methods for delivering nucleic acid molecules are well known in this art, including, but are not limited to, encapsulation in liposomes, iontophoresis, or by incorporation into other vesicles, such as biodegradable polymers, hydrogel, or cyclodextrins.

All ASO molecules used in this invention are listed in Table 1. The modified ASOs of this invention (i.e., ASO-LNAs or ASO-MOEs) were obtained by modifying the corresponding ASOs as listed in Table 1 with at least one LNA molecule or 2′-MOE modified sugar. Table 2 lists the modified ASOs molecules of this invention.

TABLE 1
The deoxyribonucleotide sequence of the present
ASOs

| | SEQ | | |
|---|---|---|---|
| | ID | | |
| Name | No. | Sequence (5′→3′) | Length |
| DCB1111128001 | 1 | GTATTTGTCTCCCAGGTCAT | 20 |
| DCB1111128002 | 2 | ACACCACGGAGCGAAGAACT | 20 |
| DCB1111128003 | 3 | TGACCACACCACGGAGCGAA | 20 |
| DCB1111128004 | 4 | CCGCTTTCCCTTGTACTGAT | 20 |
| DCB1111128005 | 5 | CTCAGTGACTCCAAATCCCG | 20 |
| DCB1111128006 | 6 | AGTGAGTGCCAACACAGTGC | 20 |
| DCB1111128007 | 7 | TTTCAGTGAGTGCCAACACA | 20 |
| DCB1111128008 | 8 | AACGAGTCAAGGTCTCTGCC | 20 |
| DCB1111128009 | 9 | GACTCGTGATGCAAAGCTGA | 20 |
| DCB1111128010 | 10 | AGACTCGTGATGCAAAGCTG | 20 |
| DCB1111128011 | 11 | ACAAGACTCGTGATGCAAAG | 20 |
| DCB1111128012 | 12 | GGAATACAAGACTCGTGATG | 20 |
| DCB1111128013 | 13 | ACCACACCACGGAGCG | 16 |
| DCB1111128014 | 14 | GCTTTCCCTTGTACTG | 16 |
| DCB1111128015 | 15 | AAAGTTCGTCTTTCGCTTGGC | 21 |
| DCB1111128016 | 16 | TAGAGCTTAAGCCTGTATGTG | 21 |
| DCB1111128018 | 17 | AACGTGCAGCTCAAAGTTGC | 20 |
| DCB1111128019 | 18 | GTTATTTTCAGTGAGTGCCA | 20 |
| DCB1111128022 | 19 | GTCTCCCAGGTCATTCCAAG | 20 |
| DCB1111128023 | 20 | AGCCACATAGACTTTGGCAT | 20 |
| DCB1111128029 | 21 | CGTTCAGTGTCTGCAGCATC | 20 |
| DCB1111128030 | 22 | TCCACTTCCGGCTCTGGTGT | 20 |
| DCB1111128031 | 23 | AAGTTGCTTGCTGAGAGCTC | 20 |
| DCB1111128032 | 24 | TGTGCAACGTGCAGCTCAAA | 20 |
| DCB1111128033 | 25 | ACGGAGCGAAGAACTTGATA | 20 |
| DCB1111128034 | 26 | TTCAAGGCCCAGAGCCAGCT | 20 |
| DCB1111128035 | 27 | TGTTCAAGGCCCAGAGCCAG | 20 |
| DCB1111128036 | 28 | TAGCCACGAACCTGGTTTCC | 20 |
| DCB1111128040 | 29 | CTTTCCCTTGTACTGATCCA | 20 |
| DCB1111128041 | 30 | CCCGCTTTCCCTTGTACTGA | 20 |
| DCB1111128042 | 31 | TGACTCCAAATCCCGCTTTC | 20 |
| DCB1111128044 | 32 | CAGTGAGTGCCAACACAGTG | 20 |
| DCB1111128045 | 33 | TATTTTCAGTGAGTGCCAAC | 20 |
| DCB1111128046 | 34 | CAAGTAGGAGCCAGAGTCTT | 20 |
| DCB1111128047 | 35 | TCCCAAGTAGGAGCCAGAGT | 20 |
| DCB1111128048 | 36 | AGAGTTCCTCCCAAGTAGGA | 20 |
| DCB1111128049 | 37 | TAGAGAGTTCCTCCCAAGTA | 20 |
| DCB1111128052 | 38 | ACTTTCTTCCCTCCTCGGAA | 20 |
| DCB1111128053 | 39 | TGACTTTCTTCCCTCCTCGG | 20 |
| DCB1111128054 | 40 | TCTCTGCCTCCACTGTGCTC | 20 |
| DCB1111128055 | 41 | AGGTCTCTGCCTCCACTGTG | 20 |
| DCB1111128056 | 42 | CGAGTCAAGGTCTCTGCCTC | 20 |
| DCB1111128057 | 43 | CCACATAGACTTTGGCATCT | 20 |
| DCB1111128058 | 44 | CCACGGAGCGAAGAACTTGA | 20 |
| DCB1111128059 | 45 | ATCCCGCTTTCCCTTGTACT | 20 |
| DCB1111128060 | 46 | TCCCAGGTCATTCCAAGTCG | 20 |
| DCB1111128064 | 47 | CAAATCCCGCTTTCCCTTGT | 20 |
| DCB1111128066 | 48 | TCCACGTACTCCCTCAGTGA | 20 |
| DCB1111128067 | 49 | AGTCTCTGTGCGCTGCAGCT | 20 |
| DCB1111128072 | 50 | CAGTCTACTTCGGCGATCTT | 20 |
| DCB1111128073 | 51 | AAGCGGTGTAACGAGTCAAG | 20 |
| DCB1111128121 | 52 | GTGAACCATACGTGATTAAT | 20 |
| DCB1111128123 | 53 | GGAACGTCAATACTGCCACA | 20 |
| DCB1111128125 | 54 | GCTTCGTGTTAACATGAGGA | 20 |
| DCB1111128132 | 55 | GAACTCTAGTTAGGGCCCTT | 20 |
| DCB1111128136 | 56 | TCCAGAACTCGTGGGCAGGT | 20 |
| DCB1111128147 | 57 | AGGATCCCCTGAACAGTAAC | 20 |
| DCB1111128148 | 58 | GGCTCTGGCTTCGTGTTAAC | 20 |
| DCB1111128149 | 59 | AGAACTCTAGTTAGGGCCCT | 20 |
| DCB1111128152 | 60 | TGGGCCAAGTATCCACATCC | 20 |
| DCB1111128171 | 61 | GCCACCTTTCCAGAACTCGT | 20 |
| DCB1111128186 | 62 | ACTGAGAATGAGCCTCGTGG | 20 |
| DCB1111128190 | 63 | CCTGGGAAAGCCTGTCTGGT | 20 |
| DCB1111128191 | 64 | GCCTCCCTGGATACCCAGGC | 20 |
| DCB1111128198 | 65 | GACATCTGTCTTTGGTCTTG | 20 |
| DCB1111128202 | 66 | GGACAGCATGAGTTAGAGGA | 20 |
| DCB1111128209 | 67 | CCTACCACCAGTTACTTTGG | 20 |
| DCB1111128217 | 68 | CTTGACAGTTGTGCTTCTCT | 20 |
| DCB1111128218 | 69 | AAGTTGTGTGTACAAGACTC | 20 |

TABLE 2
The deoxyribonucleotide sequence of ASO-LNAs or ASO-MOEs

| | SEQ ID | |
|---|---|---|
| Name | No. | Strand sequence of ASO-LNA or ASO-MOE |
| DCB1111128252 | 70 | PS-d(G_{MOE}T_{MOE}A_{MOE}T_{MOE}T_{MOE}TGTCTCCCAGG_{MOE}T_{MOE}C_{MOE}A_{MOE}T_{MOE}) |
| DCB1111128230 | 71 | PS-d(A_{MOE}C_{MOE}A_{MOE}C_{MOE}C_{MOE}ACGGAGCGAAG_{MOE}A_{MOE}A_{MOE}C_{MOE}T_{MOE}) |
| DCB1111128255 | 72 | PS-d(T_{MOE}GMORA_{MOE}C_{MOE}C_{MOE}ACACCACGGAG_{MOE}C_{MOE}G_{MOE}A_{MOE}A_{MOE}) |
| DCB1111128235 | 73 | PS-d(C_{MOE}C_{MOE}G_{MOE}C_{MOE}T_{MOE}TTCCCTTGTAC_{MOE}T_{MOE}GMORA_{MOE}T_{MOE}) |
| DCB1111128237 | 74 | PS-d(C_{MOE}T_{MOE}C_{MOE}A_{MOE}G_{MOE}TGACTCCAAAT_{MOE}C_{MOE}C_{MOE}C_{MOE}G_{MOE}) |
| DCB1111128238 | 75 | PS-d(A_{MOE}G_{MOE}T_{MOE}GMORA_{MOE}GTGCCAACACA_{MOE}G_{MOE}T_{MOE}G_{MOE}C_{MOE}) |
| DCB1111128240 | 76 | PS-d(T_{MOE}T_{MOE}T_{MOE}C_{MOE}A_{MOE}GTGAGTGCCAA_{MOE}C_{MOE}A_{MOE}C_{MOE}A_{MOE}) |
| DCB1111128251 | 77 | PS-d(A_{MOE}A_{MOE}C_{MOE}G_{MOE}A_{MOE}GTCAAGGTCTC_{MOE}T_{MOE}G_{MOE}C_{MOE}C_{MOE}) |
| DCB1111128266 | 78 | PS-d(GMOFA_{MOE}C_{MOE}I_{MOE}C_{MOE}GTGATGCAAAG_{MOE}C_{MOE}I_{MOE}GMORA_{MOE}) |
| DCB1111128277 | 79 | PS-d(A_{MOE}GMORA_{MOE}C_{MOE}T_{MOE}CGTGATGCAAA_{MOE}G_{MOE}C_{MOE}T_{MOE}G_{MOE}) |
| DCB1111128278 | 80 | PS-d(A_{MOE}C_{MOE}A_{MOE}A_{MOE}GMOFACTCGTGATGC_{MOE}A_{MOE}A_{MOE}A_{MOE}G_{MOE}) |
| DCB1111128265 | 81 | PS-d(G_{MOE}GMORAMOFA_{MOE}T_{MOE}ACAAGACTCGT_{MOE}G_{MOE}A_{MOE}T_{MOE}G_{MOE}) |
| DCB1111128280 | 13 | PS-d(A_{LNA}C_{LNA}C_{LNA}ACACCACGGAG_{LNA}C_{LNA}G_{LNA}) |
| DCB1111128279 | 14 | PS-d(G_{LNA}C_{LNA}T_{LNA}TTCCCTTGTAC_{LNA}T_{LNA}G_{LNA}) |
| DCB1111128281 | 82 | PS-d(G_{MOE}T_{MOE}C_{MOE}T_{MOE}C_{MOE}ACGCTACTGTT_{MOE}C_{MOE}T_{MOE}T_{MOE}C_{MOE}) |

Accordingly, a further aspect of this invention relates to the use of the single-stranded deoxyribonucleic acid of this invention for the manufacture of a medicament for the treatment of a disease associated with upregulation of TXNDC5, such as aging, arthritis (e.g., rheumatoid arthritis), cancer, diabetes (e.g., Type II diabetes), neurodegenerative disease, pulmonary fibrosis, kidney fibrosis, myocardial fibrosis, liver fibrosis, atherosclerosis, vitiligo, and virus infection. Examples of the cancer that may be treated by the single-stranded ASO of this invention include, but are not limited to, breast cancer, cervical cancer, colon cancer, colorectal cancer, esophageal cancer, gastric cancer, liver cancer, lung cancer, multiple myeloma, non-small cell lung cancer, pancreatic cancer, prostate cancer, renal cancer, and uterine carcinomas. Examples of the neurodegenerative disease that may be treated by the single-stranded ASO of this invention include, but are not limited to, amyotrophic lateral sclerosis, multiple sclerosis, Parkinson's disease, Alzheimer's disease, Huntington's disease, and prion disease. In one preferred example, the single-stranded ASO of this invention is for the manufacture of a medicament for the treatment of pulmonary fibrosis, kidney fibrosis, liver fibrosis, or myocardial fibrosis.

Accordingly, a pharmaceutical composition for treatment of, or prophylaxis against, the disease mediated through upregulation of TXNDC5 is provided. The pharmaceutical composition comprises at least one single-stranded deoxyribonucleic acid of this invention as an active ingredient; and a pharmaceutically acceptable carrier. Optionally, the pharmaceutical composition may further comprise another agent suitable for facilitating treatment of said disease, such as an anti-diabetic agent for the treatment of diabetic, a chemotherapeutic agent for the treatment of a cancer, a nonsteroidal anti-inflammatory drug (NSAID) for the treatment of arthritis, an anti-fibrotic medication such as nintedanib or pirfenidone for fibrotic lung disease, just to name a few.

The nucleic acid of this invention may be suspended in a suitable dispersion medium, such as water, PBS, saline, oils, or fatty acids. The pharmaceutical compositions thus prepared may be administered parenterally, by inhalation spray, topically, rectally, nasally, buccally or vaginally. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the composition is administered intramuscularly, intraperitoneally or intravenously, and most preferably, the composition is administered intramuscularly. In one example, the composition of this invention is injected intramuscularly from a site on one limb (i.e., arm or leg) of the subject. The body portion suitable for injection is selected based on the followings, such as the choice of the nucleic acid to be released, the subject's personal condition including sex, age, body weight, and/or current and prior medical conditions. An experienced physician may determine suitable body portion for injection without undue experiment. Sterile injectable forms of the composition of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, phosphate buffer solution and isotonic sodium chloride solution (i.e., saline). In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The dosage required depends on the choice of the route of administration; the nature of the formulation; the nature of the subject's illness; the subject's weight, surface area, age and sex; other drugs being administered; and the judgement of the attending physician. Suitable dosages are from 0.15 mg to 1.5 mg nucleic acid/Kg of body weight, such as 0.15, 0.2, 0.3, 0.4, 0.5, 0.6, 0.7, 0.8, 0.9, 1.0, 1.1, 1.2, 1.3, 1.4, and 1.5 mg nucleic acid/Kg of body weight; preferably from 0.3 mg to 1.2 mg nucleic acid/Kg of body weight, such as 0.3, 0.4, 0.5, 0.6, 0.7, 0.8, 0.9, 1.0, 1.1, and 1.2 mg nucleic acid/Kg of body weight; and more preferably from 0.5 mg to 1.0 mg nucleic acid/Kg of body weight, such as 0.5, 0.6, 0.7, 0.8, 0.9, and 1.0 mg nucleic acid/Kg of body weight. Variations in the needed dosage are to be expected in view of the different efficiencies of various routes for administration. Those of skill in the art can readily evaluate relevant factors and based on this information, determine the dosage to be used for an intended purpose.

This invention also features methods for treating a subject displaying an upregulation of TXNDC5, said method comprising administering the single-stranded deoxyribonucleic acid of this invention or the composition of this invention to a subject in need thereof; and further comprising administering additional medicament (e.g., a chemotherapeutic agent for the treatment of cancer) to the subject. A subject herein refers to a human and a non-human animal. In a preferred example, the subject is a human. In one example, the subject has been diagnosed with pulmonary fibrosis. In another example, the subject has been diagnosed with a cancer. In still another example, the subject has been diagnosed with rheumatoid arthritis. The subject may have received medical treatment before being subjected to the method and/or composition of this invention. In the case of cancer, the medical treatment refers to surgery, chemotherapy or radiotherapy commonly applied to a patient with tumor; therefore, to augment the antitumor effects of gene therapy, the subject pre-diagnosed with cancer may also receive other anti-tumor therapy before, at the same time or after subjecting to methods and/or compositions of this invention. In one example, the subject has pulmonary fibrosis and has been treated with pirfenidone or nintedanib before receiving the method and/or composition of this invention.

The following Examples are provided to illustrate certain aspects of the present invention and to aid those of skilled in the art in practicing this invention. These Examples are in no way to be considered to limit the scope of the invention in any manner.

### EXAMPLES

### Materials and Methods

### Cell Culture

Primary adult human pulmonary fibroblasts (HPF-a) (ScienCell, CA, USA) were cultured in Fibroblast Medium supplemented with 2% fetal bovine serum (FBS), 1% fibroblast growth supplement (FGS) and 1% penicillin/streptomycin solution; and maintained at 37° C. in a humidified environment containing 95% O₂/5% CO₂.

### Production of the Present ASOs

The human TXNDC5 mRNA were used as the target sequence for the preparation of the present ASOs. Specifically, ASOs of the present disclosure were prepared using target sequence at positions 337 to 356, 670 to 689, 675 to 694, 862 to 881, 879 to 898, 1003 to 1022, 1007 to 1026, 1278 to 1297, 2864 to 2883, 2865 to 2884, 2868 to 2887 and 2873 to 2892, respectively.

All of the ASOs of the present disclosure were obtained from Eurogentec or synthesized by using an AKTA OligoPilot 10 Plus synthesizer. The ASOs were purified by reverse phase HPLC or IEX HPLC. The purity of ASOs were analysed by UPLC. The characterization of ASOs were analysed by MOLDI-TOF or LC-HRMS.

In addition, ASOs were independently used for the preparation of modified ASOs that contained locked nucleic acid (LNA) molecules (hereafter “ASO-LNA)) or 2′-O-methoxyethyl sugar (hereafter “ASO-MOE”) modifications. Each modified ASO was synthesized in 1 nmol scale on a MOSS Expedite instrument platform accordance with the procedures described in the instrument manual.

### Transfecting HPF-a Cells With the Present ASOs

HPF-a cells were maintained and cultured in 6-well plates at the density of 1×10⁵ cells/well, and were transfected with the present ASOs described above with the aid of TransIT-X2 (Mirus Bio, USA). Specifically, plasmids contained the present ASOs and TransIT-X2 were mixed in serum-free Opti-MEM (Thermo Fisher Scientific, USA) and were added to HPF-a cell culture medium at a final concentration of 0.4-60 nM ASO and 3.3 μL TransIT-X2/1 mL medium and further incubated for another 24 hrs. The transfection of human TXNDC5 mRNA ASOs in cells was confirmed by the detection of human TXNDC5 mRNA gene expression either in RNA level by Quantitative Real Time PCR (qRT-PCR) analysis or in protein level by immunoblot assay.

Quantitative RT-PCR (qRT-PCR)

Total RNA of cells transfected with the present ASOs as described above was isolated using Direct-zol™ RNA MiniPrep kit according to the manufacturer's instructions (ZYMO Research, USA). An amount of 100 ng of DNase-treated total RNA was used as template for first strand DNA synthesis in 20 μL reaction with 4×TaqMan™ Fast 1-step master mix (Thermo Fisher Scientific, USA) containing TaqMan™ assay probe sets (hTXNDC5:Hs01046710_m1(FAM); hGAPDH:Hs03929097_g1(VIC)), which was performed in an Applied Biosystems 7500 Fast instrument that ran the following program: 50° C. for 5 min.; 95° C. for 20 sec., 40 cycles of 95° C. for 15 sec, followed by 60° C. for 1 min. The expression level of each individual transcript was normalized to control gene GAPDH and expressed relative to the mean expression values of control samples.

### Immunoblot Assay

After transfection of HPF-a cells for 24 hrs, cells were exchanged to serum free medium and treated with TGFβ1 (PeproTech, USA) at 10 ng/ml for 48 hrs. HPF-a cells were homogenized using 2× sample buffer (BioRad Laboratories, USA), followed by boiling at 95° C., 10 min. Protein samples were fractionated on 10% SDS-PAGE gel, transferred onto PVDF membrane and then blocked by blocking buffer (Visual Protein, Taiwan, BP01-IL). Membranes were incubated with primary antibodies against COL1A1 (1:500, OriGene, USA, TA309096, for human species), Fibronectin (1:2000, BD Biosciences, USA, 610077), TXNDC5 (1:15000, Proteintech, USA, 19834-1-AP), αSMA (1:1000, Abcam, UK, ab5694), β-actin (1:1000, Millipore, Germany, MAB1501) overnight at 4° C. Blots were developed using HRP-conjugated anti-mouse or anti-rabbit IgG secondary antibodies (1.5000, Cell signaling Technology, USA, 7076, 7074) and SuperSignal West Pico or Femto Chemiluminescent Substrate (Thermo Fisher Scientific, USA, 34080, 34094). Protein band detection was performed using ChemiDoc MP system (BioRad Laboratories, USA). Protein band intensity quantification analysis was performed with ImageLab software version 5.2.1.

### Bleomycin-Induced Lung Fibrosis Animal Model

8 to 9 weeks old male C57BL/6 mice were quarantined for one week after purchased. The mice were kept in individually ventilated cage systems at constant temperature and humidity with 5 animals in each cage. The room was on a 12-h light/12-h dark cycle (07:00 on and 19:00 off) and room temperature was at 22±2° C. with 55±10% humidity. Animals were allowed to access to rodent pellet food and water ad libitum. The animal experiment was performed according to the ethical rules in the National Institute of Health (NIH) Guidance for the Care and Use of Laboratory Animals.

To induce lung fibrosis, mice were intratracheally injected with Bleomycin (dissolved in sterile saline) at a dose of 3 U/kg body weight. Mice in sham group received the same volume of sterile saline only. Seven days after Bleomycin induction, mice were randomly divided into 5 groups and administrated with vehicle solution, Nintedanib, the present modified ASOs of SEQ ID NO: 73 (or DCB1111128235), SEQ ID NO: 14 (or DCB1111128279), or SEQ ID NO: 82 (or DCB1111128281) on the same study day, each group consisted of 6 mice. Nintedanib was dissolved in 10% final volume of dimethylformamide (DMF) in PBS to the final concentration of 6 mg/mL, and was administered at 60 mg/kg body weight (mpk) by oral gavage (PO) once per day (QD) for 14 days. Each of the present modified ASOs (i.e., DCB1111128235, DCB1111128279, or DCB1111128281) was dissolved in TruboFect Transfection reagent (Thermo Scientific, Mass., USA), and was administered at 0.2 mg/kg body weight (mpk) by intratracheal instillation twice per week (BIW) for 2 weeks. Mice in vehicle group received the same volume of TruboFect Transfection reagent only and served as the control group. Study was terminated on day 21 after disease induction, and lung function was determined, and lung tissues were collected and stored appropriately until analysis.

### Lung Function Tests

Lung function was assessed using the flexi Vent system (Scireq, Montreal, QC, Canada). Mice were tracheostomized and ventilated at a rate of 150 breaths/min, tidal volume of 10 ml/kg, and a positive end-expiratory pressure of 2-3 cm H₂O. A deep inflation perturbation was used to estimate the inspiratory capacity. Pressure-Volume loops were generated by constant increasing pressure, followed by regular decreasing pressure. Other lung function parameters including air resistance, compliance and elastance were measured by using SnapShot-150. Note that compliance is a factor that reflects the lung's ability to stretch and expand; air resistance is a factor that reflects the change in transpulmonary pressure needed to produce a unit flow of gas through the airways of the lung, and is the pressure difference between the mouth and alveoli of the lung divided by airflow; and elastance is a factor that reflects the pressure required to inflate the lungs.

### Pathologic Evaluation

Mice left lungs were fixed in buffered formalin and embedded in paraffin. Sections (5 μm) were stained with hematoxylin and eosin stain, or picrosirius red (Abcam, Cambridge, UK). Measurement of fibrotic area by picrosirius red stain was quantified using ImageJ software.

### Example 1 Inhibition of the Transcription of TXNDC5 mRNA by the Present ASOs

HPF-a cells were treated with the designated ASO, then the expression of TXNDC5 mRNA was measured by qRT-PCR in accordance with procedures described in the “Materials and Methods” section. Results are summarized in Table 3, in which TXNDC5 mRNA expression inhibitory activity of the designated ASO greater than 50% at 30 nM is graded as follows: +++, TXNDC5 mRNA level less than 50%; ++, TXNDC5 mRNA level between 70% and 50%.

TABLE 3
In vitro inhibition of Human TXNDC5
mRNA via the present ASOs

| | SEQ | TXNDC5 mRNA |
|---|---|---|
| DCB Ser. No. | ID No. | inhibitory activity |
| DCB1111128001 | 1 | +++ |
| DCB1111128002 | 2 | ++ |
| DCB1111128003 | 3 | +++ |
| DCB1111128004 | 4 | +++ |
| DCB1111128005 | 5 | ++ |
| DCB1111128006 | 6 | +++ |
| DCB1111128007 | 7 | ++ |
| DCB1111128008 | 8 | +++ |
| DCB1111128009 | 9 | +++ |
| DCB1111128010 | 10 | +++ |
| DCB1111128011 | 11 | ++ |
| DCB1111128012 | 12 | ++ |
| DCB1111128013 | 13 | − |
| DCB1111128014 | 14 | − |
| DCB1111128015 | 15 | ++ |
| DCB1111128016 | 16 | ++ |
| DCB1111128018 | 17 | ++ |
| DCB1111128019 | 18 | ++ |
| DCB1111128022 | 19 | ++ |
| DCB1111128023 | 20 | ++ |
| DCB1111128029 | 21 | +++ |
| DCB1111128030 | 22 | ++ |
| DCB1111128031 | 23 | +++ |
| DCB1111128032 | 24 | +++ |
| DCB1111128033 | 25 | ++ |
| DCB1111128034 | 26 | ++ |
| DCB1111128035 | 27 | ++ |
| DCB1111128036 | 28 | +++ |
| DCB1111128040 | 29 | ++ |
| DCB1111128041 | 30 | +++ |
| DCB1111128042 | 31 | +++ |
| DCB1111128044 | 32 | +++ |
| DCB1111128045 | 33 | +++ |
| DCB1111128046 | 34 | +++ |
| DCB1111128047 | 35 | ++ |
| DCB1111128048 | 36 | +++ |
| DCB1111128049 | 37 | ++ |
| DCB1111128052 | 38 | +++ |
| DCB1111128053 | 39 | +++ |
| DCB1111128054 | 40 | ++ |
| DCB1111128055 | 41 | +++ |
| DCB1111128056 | 42 | +++ |
| DCB1111128057 | 43 | +++ |
| DCB1111128058 | 44 | +++ |
| DCB1111128059 | 45 | +++ |
| DCB1111128060 | 46 | ++ |
| DCB1111128064 | 47 | ++ |
| DCB1111128066 | 48 | ++ |
| DCB1111128067 | 49 | +++ |
| DCB1111128072 | 50 | ++ |
| DCB1111128073 | 51 | ++ |
| DCB1111128121 | 52 | ++ |
| DCB1111128123 | 53 | ++ |
| DCB1111128125 | 54 | +++ |
| DCB1111128132 | 55 | ++ |
| DCB1111128136 | 56 | ++ |
| DCB1111128147 | 57 | ++ |
| DCB1111128148 | 58 | ++ |
| DCB1111128149 | 59 | ++ |
| DCB1111128152 | 60 | ++ |
| DCB1111128171 | 61 | ++ |
| DCB1111128186 | 62 | ++ |
| DCB1111128190 | 63 | ++ |
| DCB1111128191 | 64 | ++ |
| DCB1111128198 | 65 | ++ |
| DCB1111128202 | 66 | ++ |
| DCB1111128209 | 67 | ++ |
| DCB1111128217 | 68 | ++ |
| DCB1111128218 | 69 | ++ |

According to the data summarized in Table 3, among the total of ASOs prepared in accordance with procedures described in the “Materials and Methods”, 26 of them as listed in Table 3 were effective in suppressing TXNDC5 expression over 50%; and 41 of them moderately suppressed the transcription of TXNDC5 mRNA with TXNDC5 mRNA level being between 70% and 50% after treatment. The rest of ASOs (i.e., ASOs other than the 69 ASOs listed in Table 3) could only mildly suppress the transcription of TXNDC5 mRNA with TXNDC5 mRNA level remained greater than 70% after treatment (data not shown).

### Example 2 Inhibition of the TXNDC5 mRNA and TGF-β Induced Fibrosis Related Proteins by the Present Modified ASOs

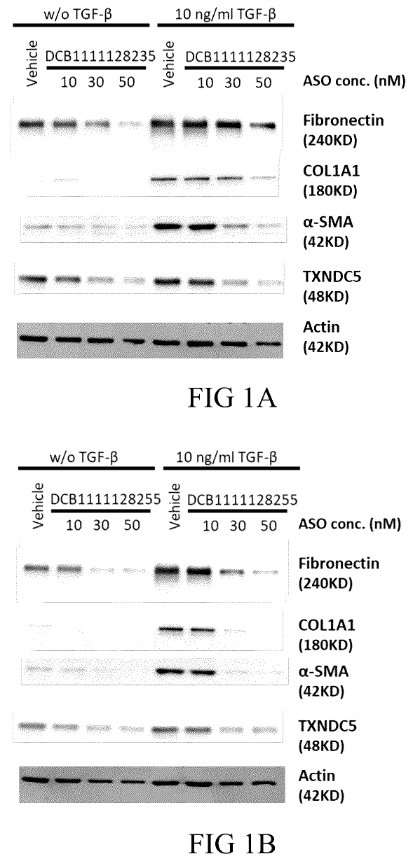

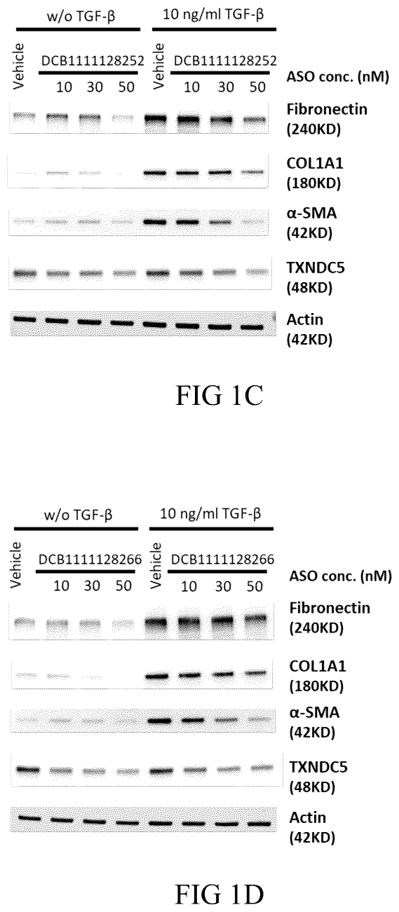

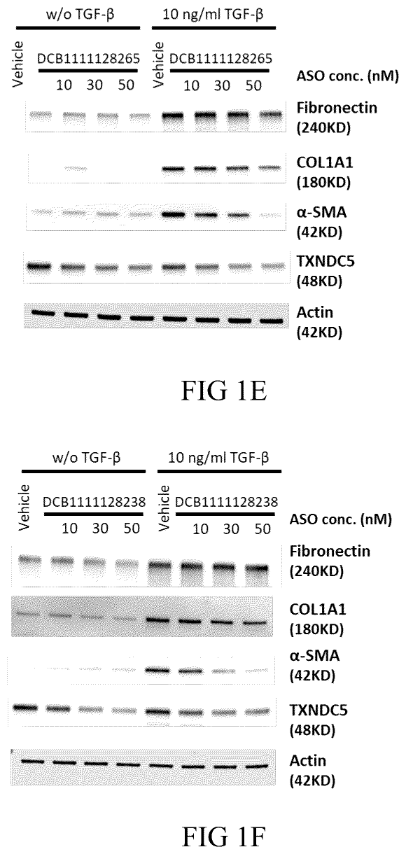

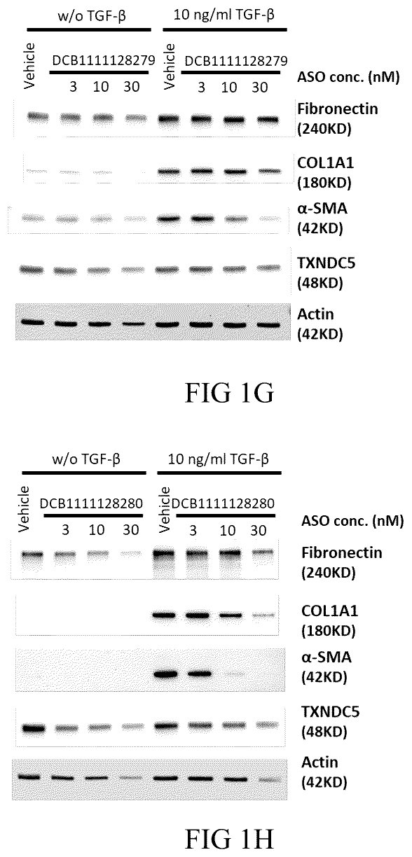

In this example, ASOs having 2′-O-methoxyethyl modified sugar (i.e., ASO-MOEs) or LNA molecules (i.e., ASO-LNAs) were derived from ASOs in Table 1 in accordance with procedures described in the “Materials and Methods” section, and their respective effects on the transcription expression level of TXNDC5 mRNA, as well as transforming growth factor beta (TGF-β) induced fibrosis related proteins were investigated. Results are summarized in Table 4 and FIG. 1.

As the data in Table 4 indicated, all the ASO-MOEs could successfully suppress the transcription of TXNDC5 mRNA with IC₅₀ below 60 nM, in which DCB1111112238 (SEQ ID NO: 75), DCB1111112240 (SEQ ID NO: 76) and DCB1111112277 (SEQ ID NO: 79) exhibited the strongest inhibitory effect with an IC50 less than 10 nM. As to ASO-LNAs, in general, it was more potent than corresponding ASO-MOE in suppressing the transcription of TXNDC5 mRNA, as IC₅₀ of ASO-LNA was smaller than that of ASO-MOE (+++ vs ++ for SEQ ID NO: 3 (or DCB1111128003) and SEQ ID No: 4 (or DCB1111128004), respectively).

TABLE 4
In vitro inhibition of TXNDC5 mRNA
by the present ASO-MOEs or ASO-LNAs

| | SEQ | TXNDC5 mRNA |
|---|---|---|
| Name | ID No. | inhibitory activity |
| DCB1111128252 | 70 | + |
| DCB1111128230 | 71 | + |
| DCB1111128255 | 72 | ++ |
| DCB1111128235 | 73 | ++ |
| DCB1111128237 | 74 | ++ |
| DCB1111128238 | 75 | +++ |
| DCB1111128240 | 76 | +++ |
| DCB1111128251 | 77 | + |
| DCB1111128266 | 78 | ++ |
| DCB1111128277 | 79 | +++ |
| DCB1111128278 | 80 | ++ |
| DCB1111128265 | 81 | ++ |
| DCB1111128280 | 13 | +++ |
| DCB1111128279 | 14 | +++ |

IC₅₀ of TXNDC5 mRNA expression inhibitory activity is graded as:
+++, IC₅₀ below 10 nM;
++, IC₅₀ level between 10 nM and 20 nM;
+, IC₅₀ level between 20 nM and 60 nM

TGF-β is known to induce expression of fibrosis related proteins, accordingly, expression of TXNDC5, and fibrosis related proteins including fibronectin, type I collagen, and α-smooth muscle actin (α-SMA) in the presence or absence of ASO-MOEs were respectively measured by immunoblot analysis. Results are illustrated in FIG. 1.

It is clear from the data in FIGS. 1A to 1I that TGF-β (10 ng/ml) would enhance the expression of fibrosis-related proteins including fibronectin, type I collagen, and α-SMA, and this enhanced protein expressions were significantly suppressed by the present ASO-MOEs including DCB11111128235 (FIG. 1A), DCB11111128255 (FIG. 1B), DCB1111128252 (FIG. 1C), DCB1111128266 (FIG. 1D), DCB1111128265 (FIG. 1E), DCB1111128238 (FIG. 1F), DCB1111128279 (FIG. 1G), DCB1111128280 (FIG. 1H), and DCB1111128281 (FIG. 11) in a dose dependent manner.

### Example 3 The Present ASO-MOEs Lessened Progression of Pulmonary Fibrosis

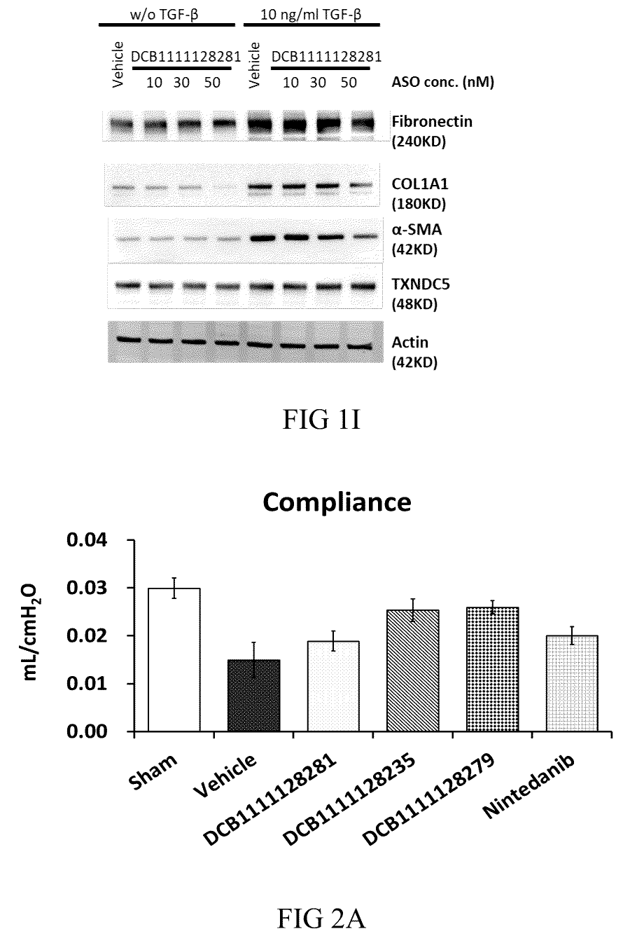

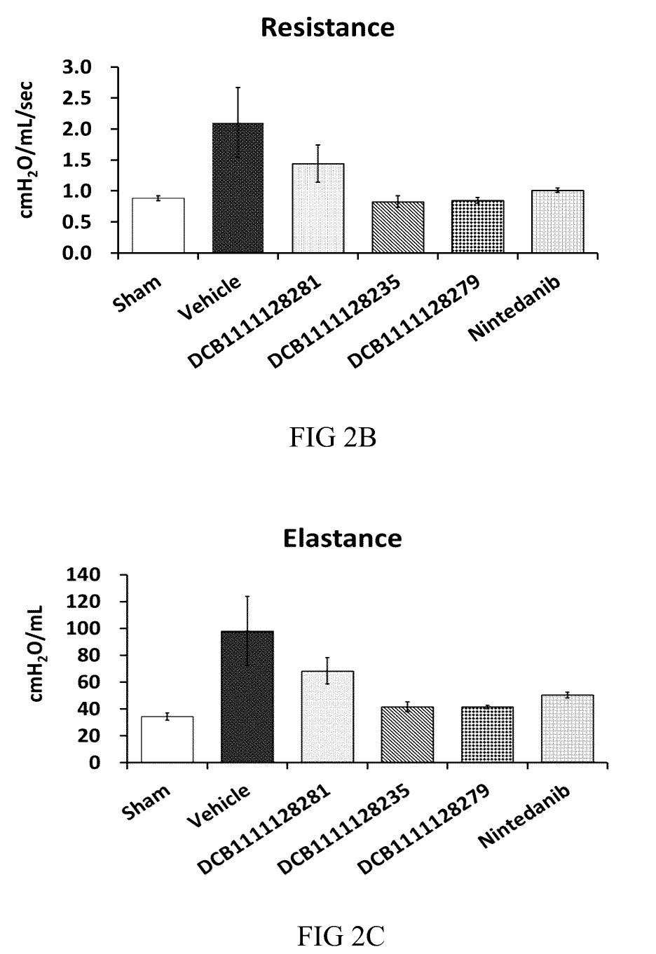

To determine the in vivo function of the present ASOs, lung fibrosis was induced by intra-tracheal instillation of Bleomycin (BLM, 3 U/Kg body weight) in accordance with the procedures described in the section of “Materials and Methods.” The fibrotic mice were then randomly divided into 5 groups (6 mice/group), in which animals in the vehicle group received PBS (intra-tracheal, 2 weeks), animals in the ASO group received the present ASO-MOE (SEQ ID Nos: 73 or 14), or scrambled ASO (SEQ ID NO: 82) (all by intra-tracheal administration route, 0.2 mg/Kg) on days 7, 10, 14, and 17, while animals in the Nintedanib group received daily dose of Nintedanib (60 mg/Kg, for 14 days). In addition, healthy animals (i.e., non-fibrotic mice) in the sham group received no treatment. Lung functions were assessed using FlexiVent system in order to determining various factors including Compliance (a factor that reflects the lung's ability to stretch and expand), Air Resistance (a factor that reflects the change in transpulmonary pressure needed to produce a unit flow of gas through the airways of the lung, and is the pressure difference between the mouth and alveoli of the lung divided by airflow), and Elastance (a factor that reflects the pressure required to inflate the lungs) on designated days, and animals were sacrificed on day 21 to collect lung samples for the determination of fibrotic area. Results are illustrated in FIGS. 2, 3, and 4.

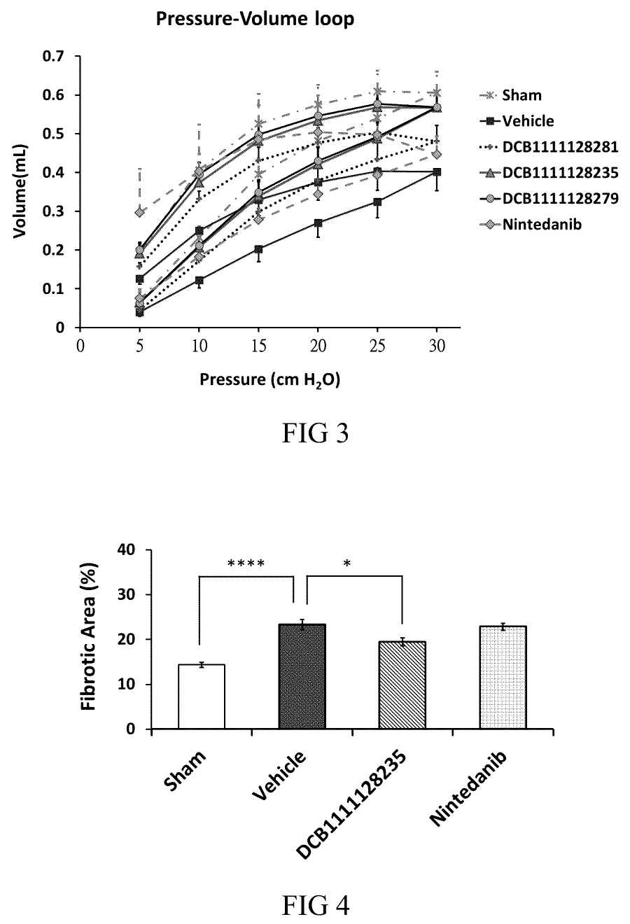

Referring to FIG. 2, which are bar graphs depicting the changes in Compliance, Resistance, and Elastance with or without being treated with the present ASO-MOE or Nintedanib. The data clearly indicates that in the case when the animals were treated with ASO-MOE of the present disclosure, the Compliance was much higher as compared with that of the vehicle control (FIG. 2A), and Resistance, and Elastance were independently much smaller as compared with that of the vehicle control (FIGS. 2B and 2C). Further, the effect of ASO-MOE (SEQ ID Nos: 73 or 14) on pressure-volume loop was more effective than that of Nintedanib (60 mg/Kg) (FIG. 3). These data indicated the fibrotic mice treated with the present ASO-MOE (SEQ ID Nos: 73 or 14) exhibited lung function (including Compliance, Resistance, Elastance, and pressure-volume loop) improvement as compared to those of the vehicle control group and the scramble ASO group (DCB1111128281 or SEQ ID NO: 82).

In terms of the effect of the present ASO-MOE on reducing BLM-induced fibrotic area in the lung tissue, it was found that the present ASO-MOE of SEQ ID No: 73 (DCB1111128235) could reduce BLM-induced fibrotic area, and its effect was more potent than that of Nintedanib (FIG. 4).

Taken together, findings in this invention support the proposition that the diseases and/disorders resulted from dysregulation of TXNDC5 such as aging, arthritis, cancer, diabetes, neurodegenerative disease, pulmonary fibrosis, vitiligo, and virus infection may be treated by introducing anti-sense oligonucleotides, particularly anti-sense oligonucleotides that interferes with the expression of TXNDC5 mRNA, into a subject (e.g., a human patient) in need of such treatment.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A single-stranded anti-sense oligonucleotide (ASO) that inhibits the translation of thioredoxin domain containing protein 5 (TXNDC5) mRNA, wherein said single-stranded ASO is about 16 to 21 nucleotides in length, and has a deoxyribonucleotide sequence that is any one of SEQ ID NOs: 1, 2, 3, 4, 5, 6, 7, 8, 9, 10, 11, 12, 13 or 14, and the deoxyribonucleotide sequence of SEQ ID NO:14 comprises 6 locked nucleic acid (LNA) molecules.

2. The single-stranded ASO of claim 1, wherein said single-stranded ASO of SEQ ID NOs: 1, 2, 3, 4, 5, 6, 7, 8, 9, 10, 11, 12 or 13 comprises at least one LNA molecule, or 2′-sugar modification.

3. The single-stranded ASO of claim 2, wherein said single-stranded ASO comprises at least one 2′-fluoro sugar, 2′-O-methyl sugar, or 2′-O-methoxyethyl sugar.

4. The single-stranded ASO of claim 2, wherein said single-stranded ASO of SEQ ID NO: 13 comprises 6 LNA molecules.

5. The single-stranded ASO of claim 3, wherein said single-stranded ASO of SEQ ID NOs: 1, 2, 3, 4, 5, 6, 7, 8, 9, 10, 11, or 12 comprises 10 2′-O-methoxyethyl sugars.

6. (canceled)

7. (canceled)

8. A method of treating a disease mediated through upregulation of thioredoxin domain containing protein 5 (TXNDC5) in a subject comprising administering to the subject an effective amount of the single-stranded ASO of claim 1 to suppress the transcription of TXNDC5 mRNA.

9. The method of claim 8, wherein said single-stranded ASO of SEQ ID NOs: 1, 2, 3, 4, 5, 6, 7, 8, 9, 10, 11, 12 or 13 comprises at least one LNA molecule, or 2′-sugar modification.

10. The method of claim 9, wherein said single-stranded ASO comprises at least one 2′-fluoro sugar, 2′-O-methyl sugar, or 2′-O-methoxyethyl sugar.

11. The method of claim 9, wherein said single-stranded ASO of SEQ ID NO: 13 comprises 6 LNA molecules.

12. The method of claim 10, wherein said single-stranded ASO of SEQ ID NOs: 1, 2, 3, 4, 5, 6, 7, 8, 9, 10, 11 or 12 comprises 10 2′-O-methoxyethyl sugars.

13. (canceled)

14. (canceled)

15. The method of claim 8, wherein the disease is selected from the group consisting of aging, arthritis, cancer, diabetes, neurodegenerative disease, fibrosis, atherosclerosis, vitiligo, and virus infection.

16. The method of claim 15, wherein the cancer is selected from the group consisting of breast cancer, cervical cancer, colon cancer, colorectal cancer, esophageal cancer, gastric cancer, liver cancer, lung cancer, multiple myeloma, non-small cell lung cancer, pancreatic cancer, prostate cancer, renal cancer, and uterine carcinomas.

17. The method of claim 15, wherein the neurodegenerative disease is selected from the group consisting of amyotrophic lateral sclerosis, multiple sclerosis, Parkinson's disease, Alzheimer's disease, Huntington's disease, and prion disease.

18. The method of claim 15, wherein the fibrosis is selected from the group consisting of pulmonary fibrosis, kidney fibrosis, liver fibrosis and myocardial fibrosis.

19. The method of claim 8, wherein the subject is a human.
